SEQUENCE LISTING (11 sequences)

SEQ ID NO: 1 - RNA, 19 nt, Artificial Sequence
  GRP 94 siRNA
ugauguggaugguacagua

SEQ ID NO: 2 - RNA, 19 nt, Artificial Sequence
  GRP 94 siRNA
uacuguaccauccacauca

SEQ ID NO: 3 - RNA, 19 nt, Artificial Sequence
  GRP 94 siRNA
gaagaagcaucugauuacc

SEQ ID NO: 4 - RNA, 19 nt, Artificial Sequence
  GRP 94 siRNA
gccuuccaagccgaaguua

SEQ ID NO: 5 - RNA, 19 nt, Artificial Sequence
  GRP 94 siRNA
ccuuccaagccgaaguuaa

SEQ ID NO: 6 - RNA, 19 nt, Artificial Sequence
  GRP 94 siRNA
ucuggaaaugaggaacuaa

SEQ ID NO: 7 - RNA, 19 nt, Artificial Sequence
  GRP 94 siRNA
ccuugguaccauagccaaa

SEQ ID NO: 8 - RNA, 19 nt, Artificial Sequence
  GRP 94 siRNA
gcaucugauuaccuugaau

SEQ ID NO: 9 - RNA, 19 nt, Artificial Sequence
  GRP 94 siRNA
ggagucugacuccaaugaa

SEQ ID NO: 10 - RNA, 19 nt, Artificial Sequence
  control siRNA
ccuacgccaccaauuucgu

SEQ ID NO: 11 - RNA, 19 nt, Artificial Sequence
  TRAF2 siRNA
caaccagaaggugaccuua

(19) United States
(12) Patent Application Publication
(10) Pub. No.: US 2010/0273144 A1
(43) Pub. Date: Oct. 28, 2010

(54) NOVEL USE OF GRP 94 IN VIRUS INFECTION

(76) Inventors: Sung-Key Jang, Gyungsangbuk-do (KR); Song-Hee Lee, Kyungsangbuk-do (KR)

Correspondence Address: JHK Law, P.O. Box 1078, La Canada, CA, 91012-1078, US

(21) Appl. No.: 12/814,413

(22) Filed: Jun. 11, 2010

Related U.S. Application Data
(62) Division of application No. 12/355,747, filed on Jan. 16, 2009.
(60) Provisional application No. 61/021,994, filed on Jan. 18, 2008.

(51) Int. Cl. C12Q 1/70

(52) U.S. Cl. 435/5

(57) ABSTRACT

A novel use of GRP 94 in treatment of virus infection is provided. More specifically, a method of inhibiting virus infection by inhibiting expression of GRP 94 and/or inactivating activity of GRP 94, and a method of developing drugs for preventing and/or treating virus infection and/or diseases caused by virus infection by using GRP 94 as a target are provided.

### CROSS-REFERENCE TO RELATED APPLICATION

The present application is a divisional of U.S. patent application Ser. No. 12/355,747, filed Jan. 16, 2009 (pending), which claims the benefit of priority to U.S. Provisional Application No. 61/021,994, filed on Jan. 18, 2008, the contents of which are incorporated by reference herein in their entirety.

### BACKGROUND OF THE INVENTION

(a) Field of the Invention

A novel use of GRP 94 in treatment of virus infection is provided. More specifically, a method of inhibiting virus infection by inhibiting expression of GRP 94 and/or inactivating activity of GRP 94, and a method of developing drugs for preventing and/or treating virus infection and/or diseases caused by virus infection by using GRP 94 as a target are provided.

(b) Description of the Related Art

About 170 million people (3% of the global population) are estimated to be infected with hepatitis C virus (HCV). Epidemiological studies have suggested that about 80% of acute HCV cases develop into chronic infections, resulting in chronic hepatitis. This high incidence of chronic HCV infection indicates that the virus produces one or more proteins that actively block the anti-viral functions of the host. The molecular bases of the pathogenic hepatic injury and the viral persistence mechanisms are, however, largely unknown.

Some viruses ensure their survival by blocking the host anti-infective apoptotic mechanisms with a variety of viral proteins that modulate various stages of the death-signaling pathways. Therefore, HCV may express a protein(s) that blocks apoptosis in the infected cells.

The transcription factor NF-κB is composed of homo- or heterodimers of polypeptides of Rel family members. Inactive NF-κB is restricted to the cytoplasm owing to its interaction with inhibitory proteins known as IκBs, which mask the nuclear translocation signal of NF-κB. Following stimulation with, for example, the proinflammatory cytokines tumor necrosis factor (TNF)-α and interleukin-1, IκBs are phosphorylated by IκB kinase (IKKα/ε), which results in proteasome-dependent degradation of IκBs. The degradation of IκBs leads to the release of NF-κB and allows NF-κB to translocate into the nucleus, where it activates the transcription of target genes. Activation of NF-κB is an immediate early step required for activation of the host immune system. Therefore, many viral proteins disrupt the innate immune responses mediated by NF-κB by nullifying signaling cascades that activate NF-κB.

Activated NF-κB also induces expression of anti-apoptotic proteins including X-chromosome-linked inhibitor of apoptosis protein (XIAP), c-IAPs (inhibitors of apoptosis proteins), c-FLIP (FLICE-like inhibitory protein), TNFα-receptor-associated factor 2 (TRAF2), and Bcl-XL, which inhibit multiple stages of apoptosis. In this respect, activation of NF-κB was shown to facilitate some viral infections by promoting viral replication, preventing virus-induced apoptosis, priming the host cell for infection, and contributing to an oncogenic transformation.

### SUMMARY OF THE INVENTION

One embodiment of the present invention relates to a method of inhibiting activity of NF-κB by inhibiting GRP 94.

Another embodiment relates to a method of inhibiting virus infection by inhibiting GRP 94.

Still another embodiment relates to a method of preventing or treating a disease caused by virus infection by inhibiting GRP 94.

Still another embodiment relates to a method of developing a drug for inhibiting virus infection, and/or preventing and/or treating a disease caused by virus infection using GRP 94 as a target.

### BRIEF DESCRIPTION OF THE DRAWINGS

FIGS. 1A to 1C show results of western blot analysis indicating that HCV E2 augments expression of NF-κB related genes products, wherein

1A shows the levels of XIAP, TRAF2, FLIP, phospho-IKKα/β (p-IKKα/β), HCV E2 (E2) and actin in control cells (lane 1) and in spE2 cells (lane 2),

1B shows the levels of XIAP, TRAF2, survivin, HCV NS5A (NS5A) and actin in control Huh-7 cells (lane 1), Huh-7 cells containing replicons FK-E1(UAA) (lane 2) and FK-E2(UAA) (lane 3), and

1C shows the results of time-course analysis of IκBα levels after TNF-α treatment (10 ng/ml) in control Huh-7 cells (lanes 1-5), Huh-7 cells containing replicons FK-E1(UAA) (lanes 6-10) and FK-E2(UAA) (lanes 11-15).

FIGS. 2A and 2B shows the level of GRP94 measured by Immunocytochemical analyses, indicating that the level of GRP94 is higher in cells expressing HCV E2, wherein

2A shows results of the immunocytochemical analyses of GRP94 and E2 in spE2 cells, and

2B shows results of the immunocytochemical analyses of GRP94 and E2 proteins in Huh-7 cells containing the replicon FK-E2(UAA).

FIGS. 3A to 3C show the results indicating that GRP94 induces NF-κB-related survival gene products, wherein

3A shows the levels of NF-κB-related survival gene products in cells overexpressing GRP94. The levels of various proteins in control cells (lane 1) and in GRP94-overexpressing cells (lane 2) were assessed by Western-blot analysis,

3B shows subcellular localizations of GRP94 and IKKα/ε in normal and GRP94-overexpressing cells, and

3C shows effects of siRNAs against GRP94 on the levels of NF-κB-related survival gene products in cells.

FIGS. 4A to 4C show that GRP94 activates NF-κB, wherein

4A shows the results of NF-κB reporter assay,

4B shows effect of abrogation of IKKα/β activity by BAY11-7082 on the expression of NF-κB-related survival genes, as monitored by Western-blot analysis, and

4C shows effect of a siRNA against TRAF2 on the expression of NF-κB-related survival genes, as monitored by Western-blot analysis.

FIG. 5 shows results of Western-blot analysis indicating that GRP94 inhibits TRAIL-induced apoptosis.

FIGS. 6A to 6D show that GRP94 is needed for efficient infection of HCV, wherein

6A is a schematic representation of JFH15a-Rluc containing the Renilla luciferase gene in NS5A of the infectious HCV strain JFH1,

6B shows the levels of GRP94 and NF-κB-related survival gene products in control and HCV (JFH 5a-Rluc)-infected Huh-7 cells, as monitored by Western-blot analysis.

6C shows results of immunocytochemical analyses of GRP94 and HCV E2 in HCV-infected cells.

6D shows effects of TRAIL and a siRNA against GRP94 on infectivity of HCV.

FIGS. 7A to 7C show that GRP94 and XIAP are overexpressed in hepatocyte cells infected with HCV, wherein

7A visualizes liver cells from a normal liver (left panel) and HCV patients (middle and right panels) by H&E staining.

7B shows the protein levels of GRP94, HCV E2, XIAP, and GAPDH in the liver cells, as monitored by Western blotting.

7C shows the results of immunohistochemical analyses of liver tissues from normal (panels a, b, g, h, m, n, s, and t), HCV-infected (c, d, I, j, o, p, u, and v), HCC (e, f, k, l, q, r, w, and x) donors.

### DETAILED DESCRIPTION OF THE EMBODIMENTS

The inventors have previously shown that HCV E2 inhibits the apoptosis of host cells by preventing activation of caspases. Here, we report that the anti-apoptotic activity of E2 is mediated by activation of NF-κB, which directs expression of survival gene products such as TRAF2, XIAP, FLIP and survivin. Increased levels of these proteins were observed in both HCV-infected cells and an HCV-E2-expressing cell line. The activation of NF-κB was mediated by HCV-E2-induced increased expression of the molecular chaperone GRP94. Overexpression of GRP94 alone resulted in expression of the anti-apoptotic proteins and blocked apoptosis induced by tumor necrosis-related apoptosis-inducing ligand (TRAIL). Interestingly, increased levels of GRP94 were observed in cells supporting HCV proliferation that originated from liver tissues of HCV patients. Moreover, siRNA-knock-down of GRP94 reduced the anti-apoptotic activity of HCV E2. These data indicate that HCV E2 blocks apoptosis induced by HCV infection and the host immune system through overproduction of GRP94, and that HCV E2 plays an important role in persistent HCV infection.

The inventors found novel uses of GRP 94 to activate NF-κB, and prevent apoptosis in virus (e.g., HCV)-infected cells, thereby facilitating persistent infection of the virus, to complete the present invention. The activity of GRP 94 to prevent apoptosis in virus-infected cells indicates that inhibition against GRP 94 results in inhibiting virus infection, and/or preventing and/or treating a disease caused by virus infection.

Hereinafter, the present invention will be described more detail.

One embodiment of the present invention provides a method of inhibiting activity of NF-κB by inhibiting GRP 94.

Another embodiment of the present invention provides a method of inhibiting virus infection by inhibiting GRP 94.

Still another embodiment of the present invention provides a method of preventing and/or treating a disease caused by virus infection by inhibiting GRP 94.

The above methods may be performed by administering an inhibitor against GRP 94 to a subject susceptible to virus infection, or in need of inhibition of NF-κB and/or virus infection, and/or prevention and/or treatment of a disease caused by virus infection. Since GRP 94 belongs to Heat Shock Protein 90 (Hsp90) family, the inhibitor may be one or more selected from Hsp90 family inhibitors, such as geldanamycin and its derivatives, and the like. In addition, the inhibitor may be one or more selected from siRNAs, antibodies, oligopeptides, and/or aptamers against GRP 94.

The derivatives of geldanamycin may be any compound derived from the natural product geldanamycin, which may includes, but not be limited to, 17-allylamino-demethoxygeldamycin (17-AAG), 17-dimethylamino-geldanamycin (17-DMAG), and the like.

The siRNA against GRP 94 may be any oligonucleotide capable of inhibiting the expression of GRP 94 coding gene, for example having the nucleotide sequence of 5′-UGA UGU GGA UGG UAC AGU A-3′ (SEQ ID NO: 1) or 5′-UAC UGU ACC AUC CAC AUC A-3′ (SEQ ID NO: 2), 5′-GAA GAA GCA UCU GAU UAC C-3′ (SEQ ID NO: 3), 5′-GCC UUC CAA GCC GAA GUU A-3′ (SEQ ID NO: 4), 5′-CCU UCC AAG CCG AAG UUA A-3′ (SEQ ID NO: 5), 5′-UCU GGA AAU GAG GAA CUA A-3′ (SEQ ID NO: 6), 5′-CCU UGG UAC CAU AGC CAA A-3′ (SEQ ID NO: 7), 5′-GCA UCU GAU UAC CUU GAA U-3′ (SEQ ID NO: 8), 5′-GGA GUC UGA CUC CAA UGA A-3′ (SEQ ID NO: 9), and the like.

The antibodies against GRP 94 may be any protein capable of antigen-antibody interaction with GRP 94. The oligopeptides against GRP 94 may be any oligopeptide capable of binding GRP 94, preferably having 4-10 amino acids. The oligopeptides may be administered by any conventional manners, such as by being conjugated with a peptide for intracellular delivery. The aptamer against GRP 94 may be any oligonucleotide capable of binding GRP 94.

One skilled in the art can readily determine an effective amount of the inhibitors to be administered to the subject, by taking into account factors such as the health, sex, age and weight of the subject; the condition of infection or relevant diseases; the route of administration; and whether the administration is regional or systemic. One skilled in the art can also readily determine the proper route of administration according to the type of the inhibitor to be administered. For example, the inhibitor may be administered orally, enterally or parenterally. In case siRNA and aptamer are employed, siRNA and aptamer may be administered as naked siRNA; in conjunction with a delivery reagent such as Mirus Transit TKO lipophilic reagent, lipofectin; lipofectamine; cellfectin; or polycations (e.g., polylysine), or liposome; or as a recombinant plasmid or viral vector which expresses the siRNA and aptamer. Alternatively, siRNA and aptamer may be administered by any means suitable for delivering (or injecting or infusing) the siRNA and aptamer to the cells of the tissue, for example by gene gun, electroporation, or by other suitable parenteral or enteral administration routes.

Due to the activity of GRP 94 to prevent apoptosis of virus-infected cells, inhibition against. GRP 94 may result in inhibiting infection of various viruses. The virus may be one or more selected from the group consisting of hepatitis viruses, and preferably hepatitis c virus (HCV), or hepatitis b virus (HBV)

As well known to the relevant arts, virus infection may cause various diseases such as inflammation, cancer, and the like. In particular, infection of hepatitis viruses may cause hepatitis, cirrhosis, liver cancer such as hepatocellular carcinoma, and the like.

Therefore, the activity of GRP 94 to prevent apoptosis in virus-infected cells may be applied to inhibiting virus infection, and/or preventing and/or treating diseases caused by virus infections such as inflammation, cancer, and the like, and in preferable embodiment, the virus may selected from hepatitis viruses including hepatitis c virus (HCV), or hepatitis b virus (HBV), and the disease may be hepatitis, cirrhosis, liver cancer such as hepatocellular carcinoma, and the like,

The subject may be any animal, preferably mammal, more preferably human, who is susceptible to virus infection or need of inhibiting virus infection, and/or preventing and/or treating diseases caused by virus infection.

Another embodiment of the present invention provides a method of developing or identifying an antiviral agent or a drug for inhibiting virus infection or preventing or treating a disease caused by virus infection using GRP 94 as a target. The method may include the following steps of:

contacting a candidate compound to a biological sample;

measuring the level of expression of GRP 94, or activity of GRP 94; and

comparing the level measured in the biological sample contacted with the candidate compound to that measured the biological sample without contacting with the candidate compound,

wherein the candidate compound is determined as the drug for inhibiting virus infection or preventing or treating a disease caused by virus infection, when the level measured in the biological sample contacted with the candidate compound is decreased compared with that measured in the biological sample without contacting with the candidate compound.

As described above, the biological sample may be any sample from animals, preferably mammal, for example including a cell, a tissue, an extract of cells or tissues, a body fluid, and the like. In a preferable embodiment, the biological sample may be hepatocytes. In another embodiment, the biological sample may be virus-infected cells or tissues.

The virus may be one or more selected from the group consisting of hepatitis viruses, preferably Hepatitis c Virus (HCV) or Hepatitis b Virus (HBV), and the disease may be one or more selected from the group consisting of inflammation, cancer, and the like, preferably hepatitis, cirrhosis, liver cancer such as hepatocellular carcinoma, and the like.

The level of expression of GRP 94 may be measured by any conventional method, including western blotting method, immunocytochemical method, ELISA, and the like, but not limited thereto. The activity of GRP 94 may be measured by any conventional method known to the relevant arts such as ATPase activity. In particular, since GRP 94 activates NF-κB, the activity of GRP 94 may be measured as the expression levels or the activities of anti-survival gene products under the control of NF-κB activity, including tumor necrosis factor (TNF-α) receptor-associated factor 2 (TRAF2), X-chromosome-linked inhibitor of apoptosis protein (XIAP), FLICE-like inhibitory protein (FLIP), surviving, and the like.

Glucose-regulated protein 94 (GRP94) is the endoplasmic reticulum (ER)-resident member of the heat-shock-protein 90 (Hsp90) family. Hsp90 and GRP94 interact with their counterparts (client proteins) and protect them from ubiquitin-dependent proteasomal degradation. Although the GRP94 protein is expressed constitutively in all cell types, its expression is up-regulated under various stress conditions including low glucose levels, low extracellular pH, expression of mutated proteins, and viral infections. Heat-shock proteins have a cytoprotective function and modulate apoptosis directly or indirectly. Previous studies have shown that expression of GRP94 is increased in tumor cells, including hepatocellular carcinoma, colorectal carcinoma and lung cancer cells, and that GRP94 has an anti-apoptotic effect on some tumor cells. Moreover, increased levels of GRP94 were observed when a chronic hepatitis B virus (HBV) infection progressed to cirrhosis and hepatocellular carcinoma (HCC). Inhibitors of Hsp90 and GRP94 [such as geldanamycin (GA) and its less toxic derivative 17-AAG] have been investigated for efficacy in cancer treatment. GA and 17-AAG inhibit Hsp90 and GRP94 by binding competitively to its N-terminal ATP-binding site. Inhibition of ATPase activity results in misfolding and degradation of client proteins through the ubiquitin-proteasome pathway. 17-AAG is undergoing phase II clinical trials for use in metastatic melanoma, prostate cancer and multiple myeloma.

GRP94 may be encoded by the following genes, but not limited thereto:

NM_{—}003299, BC066656: Homo sapiens heat shock protein 90 kDa beta (Grp94), member 1, mRNA (cDNA clone MGC:75130 IMAGE:6165138), complete cds;

NM_{—}011631: Mus musculus heat shock protein 90, beta (Grp94), member 1 (Hsp90b1), mRNA;

NM_{—}001045763: Xenopus (Silurana) tropicalis heat shock protein 90 kDa beta (Grp94), member 1 (hsp90b1), mRNA;

NM_{—}214103: Sus scrofa heat shock protein 90 kDa beta (Grp94), member 1 (HSP90B1), mRNA;

NM_{—}198210: Danio rerio heat shock protein 90, beta (grp94), member 1 (hsp90b1);

NM_{—}001012197: Rattus norvegicus tumor rejection antigen gp96 (Tra1), mRNA;

NM_{—}001134101: Pongo abelii heat shock protein 90 kDa beta (Grp94), member 1 (HSP90B1);

NM_{—}001003327: Canis lupus familiaris heat shock protein 90 kDa beta (Grp94), member 1 (HSP90B1), mRNA;

NM_{—}204289:Gallus gallus heat shock protein 90 kDa beta (Grp94), member 1 (HSP90B1), mRNA;

DQ662235:Paralichthys olivaceus glucose-regulated protein 94 (Grp94) mRNA, complete cds; and the like.

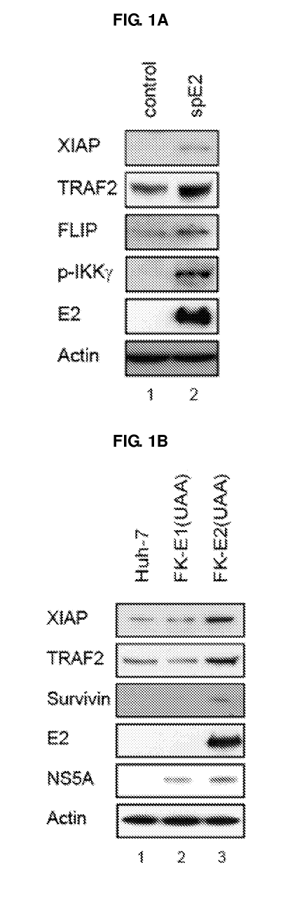

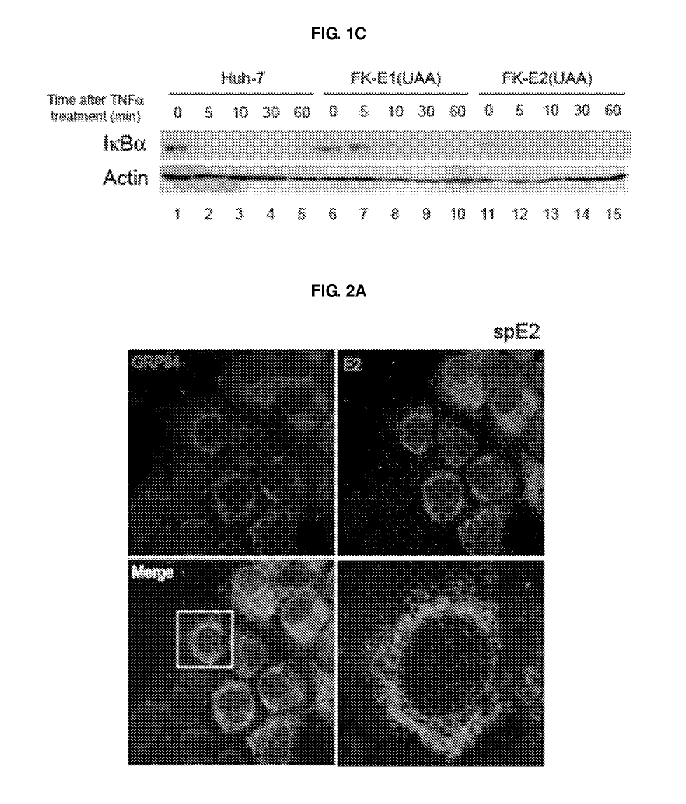

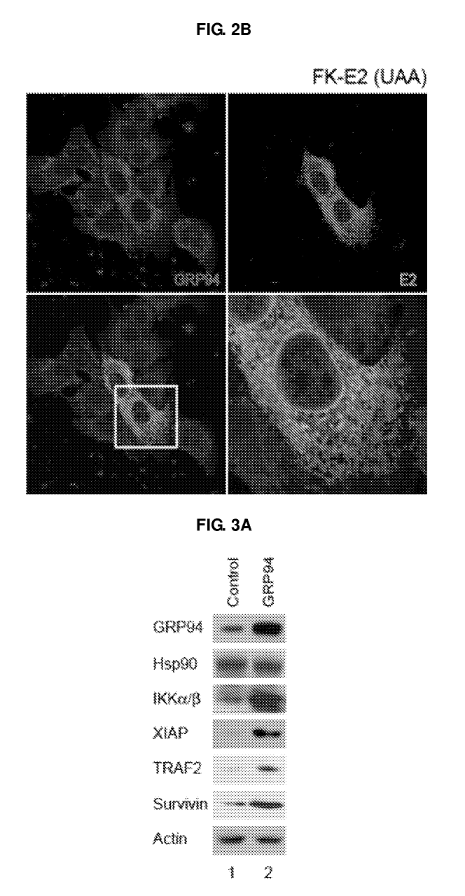

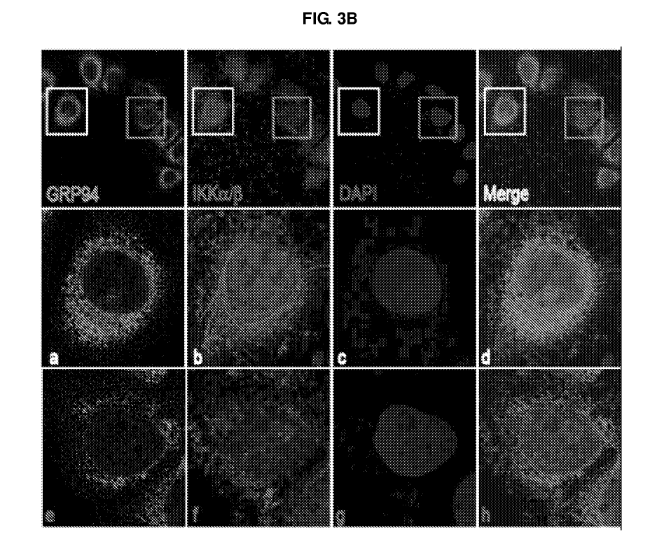

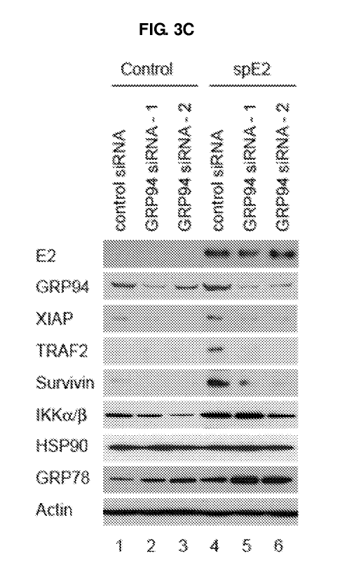

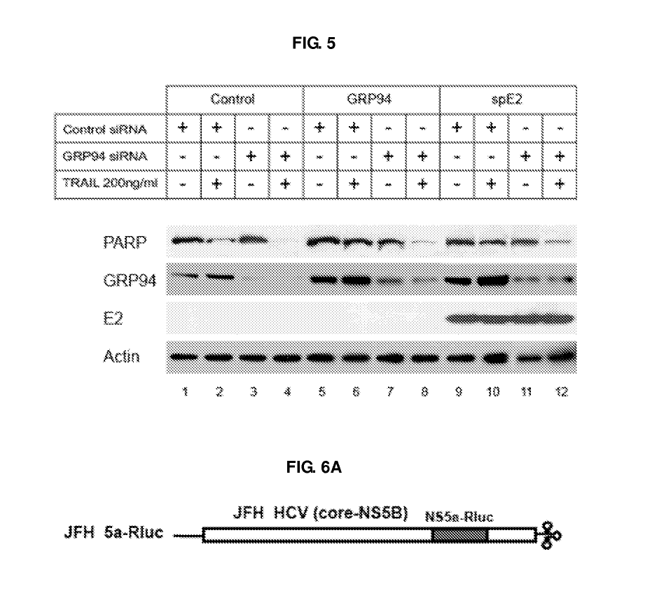

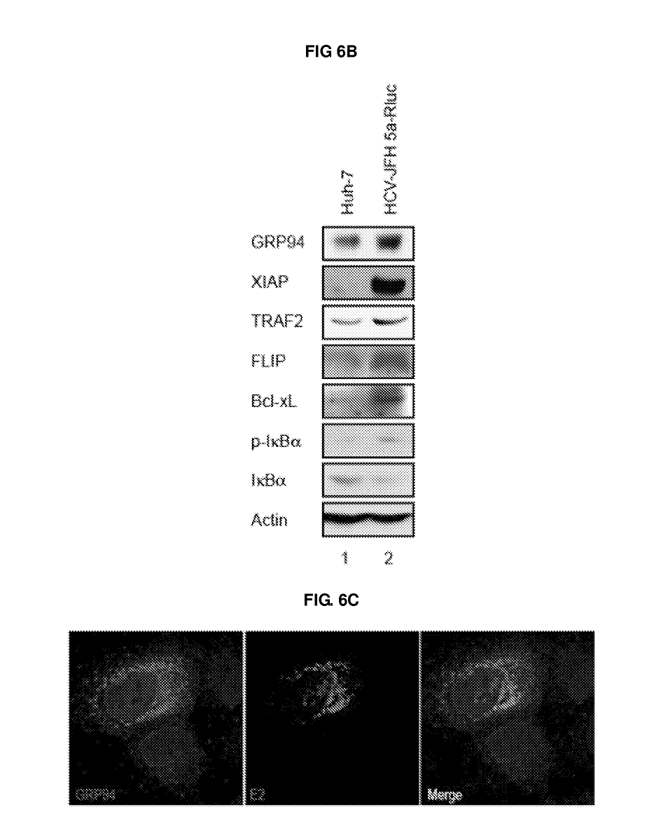

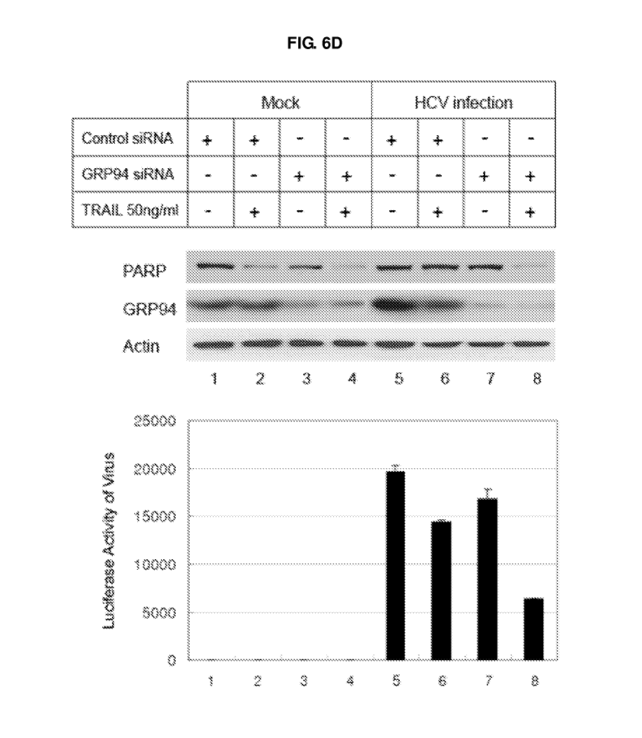

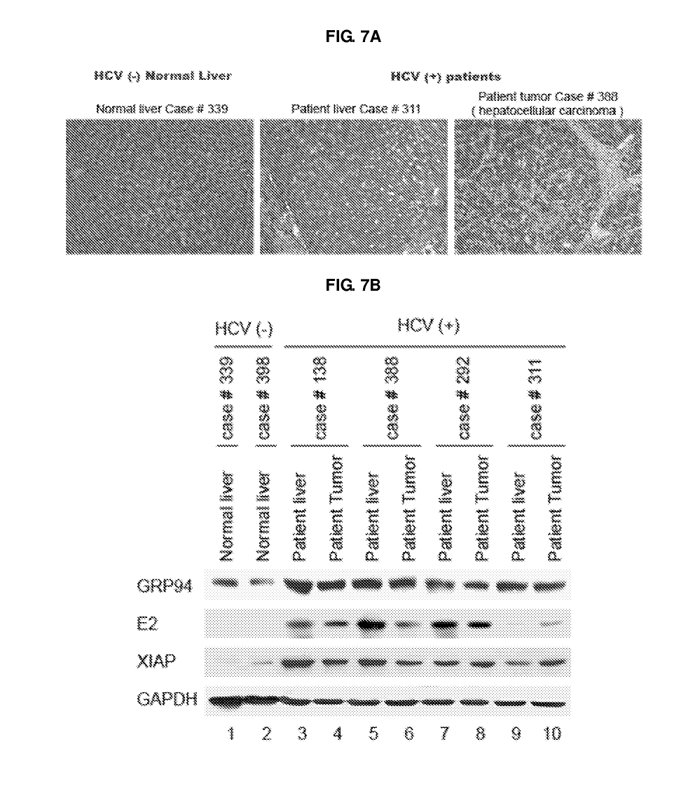

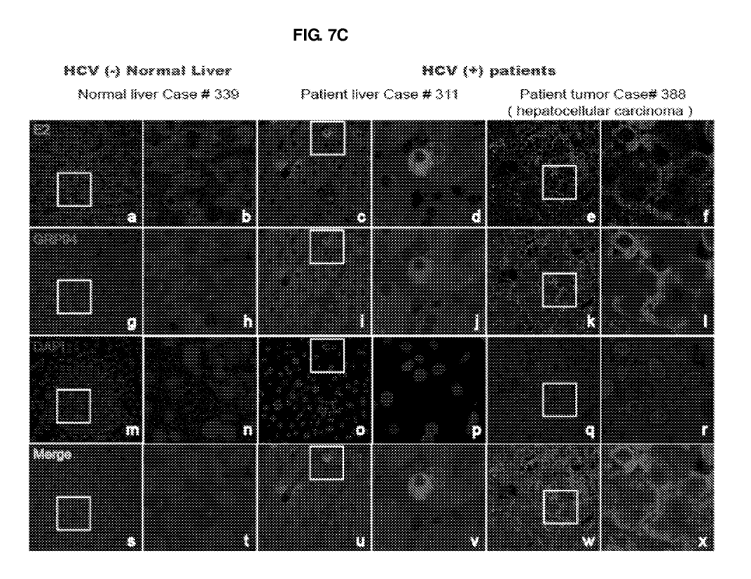

Many viruses encode proteins that suppress or delay apoptosis of host cells long enough for the viruses to replicate or to establish persistent infection. The HCV envelope protein E2 has been shown to have anti-apoptotic activity and to augment colony formation by HCV replicons. Here, we report the molecular basis of the anti-apoptotic activity of HCV E2. Several lines of evidence indicate that the anti-apoptotic activity of HCV E2 is mediated by overproduction of the molecular chaperone GRP94. First, HCV E2 induces overproduction of GRP94 (FIG. 2), as previously described by Liberman et al. Second, both HCV-E2-expressing cells (spE2) and GRP94-overexpressing cells induced expression of NF-κB-related anti-apoptotic gene products such as XIAP, FLIP and survivin (FIGS. 1A, 1B and 3A). Third, the expression of NF-κB-related gene products in HCV-E2-expressing cells was reduced by the knockdown of GRP94 with siRNAs (FIG. 3C). Fourth, GRP94 was induced in cells infected with the HCV strain JFH 5a-Rluc (FIGS. 6B and 6C). Moreover, marked induction of anti-apoptotic proteins such as XIAP, FLIP and Bcl-xL, which are under the control of NF-κB, was observed in the HCV-infected cells (FIG. 6B). The induction of GRP94 by HCV infection was also seen in the liver tissues of HCV patients (FIG. 7C). Finally, TRAIL-induced apoptosis was inhibited in E2-expressing cells and GRP94-overexpressing cells (FIG. 5). Importantly, the anti-apoptotic activity of E2 was nullified by treatment of E2-overexpressing cells or HCV-infected cells with a siRNA against GRP94 (FIG. 5; FIG. 6D). Taken together, these data indicate that HCV E2 facilitates persistent infection of HCV by inducing GRP94.

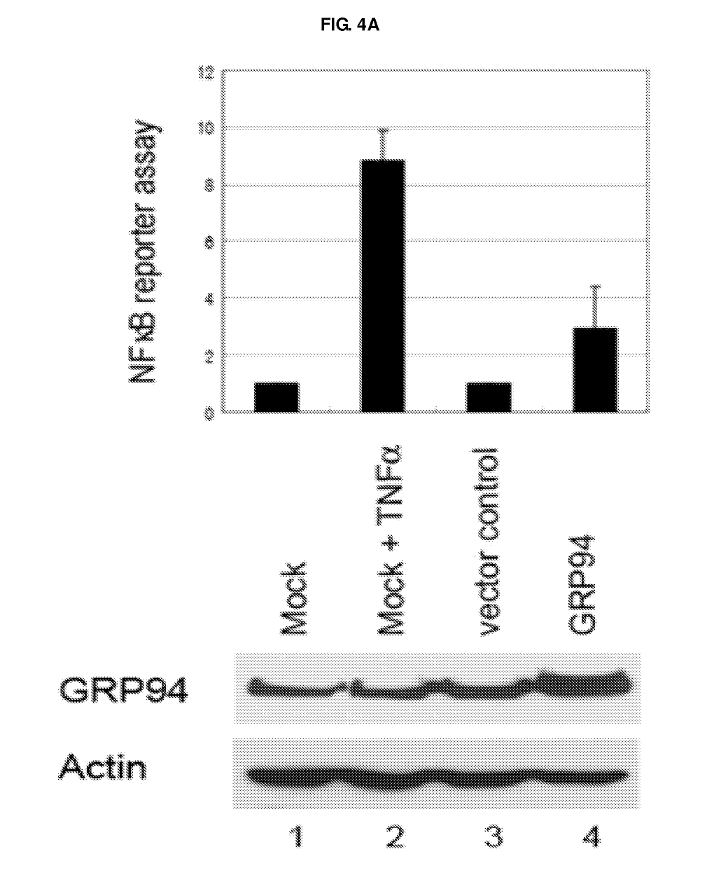

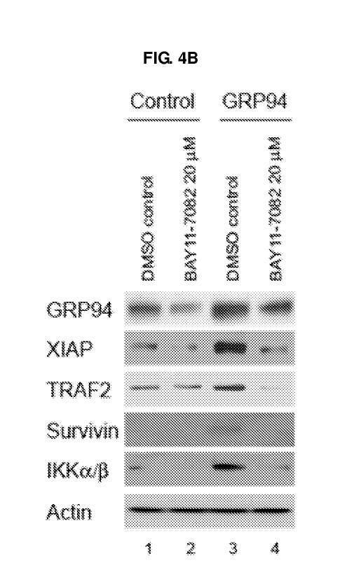

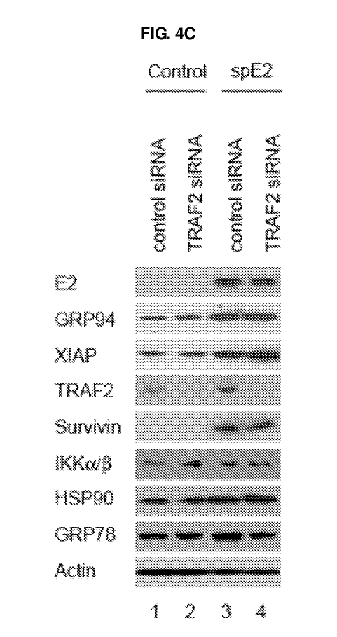

The increased level of GRP94 results in production of anti-apoptotic proteins such as XIAP, FLIP and survivin (FIGS. 1A and 1B). It is most likely that these survival-related genes are induced by activation of NF-κB because these genes are under the transcriptional control of NF-κB. Moreover, expression of these proteins requires the activation of IKKα/β (FIG. 4B) and the reduction of IκBα level (FIG. 1C), both of which are prerequisite events for NF-κB activation. The molecular basis of GRP94-induced NF-κB activation is not fully understood. Nevertheless, it seems to be clear that IKKα/β activation plays a key role in the GRP94-medicated activation of NF-κB because an IKK inhibitor (BAY11-7082) blocked NF-κB activation by GRP94 (FIG. 4B). It should be noted that the IKKα/β proteins were redistributed in the GRP94-overexpressing cells and were partially co-localized with GRP94 (FIG. 3B). Moreover, IKKα/β proteins were co-immunoprecipitated with GRP94. This may indicate that GRP94 forms a complex with IKKα/β and activates IKKα/β directly or indirectly through an unidentified protein. It remains unclear how a protein in the ER lumen (GRP94) can activate IKKα/β, which is on the cytosolic side of the ER membrane. In this respect, it should be noted that GRP94 is known to exist at multiple loci (cell surface, transmembrane and ER lumen). The GRP94 in the membrane may participate in activation of IKKα/β. TRAF2, which plays a key role in TNF-α-receptor-mediated NF-κB activation does not seem to be involved in the NF-κB activation mediated by GRP94 because a siRNA against TRAF2 does not hamper the NF-κB activation by GRP94 (FIG. 4C). The molecular detail of the mechanism by which GRP94 activates IKKα/β remains to be determined.

Two other HCV proteins are known to activate NF-κB. Core and NS5A were shown to activate NF-κB through distinct pathways. However, the activities of these proteins were not sufficiently strong to trigger induction of survival genes such as XIAP and survivin, as shown in cells containing the FK-E1(UAA) replicon, which produces core and NS5A but not E2 (FIG. 1B). On the other hand, these genes were induced in cells containing the FK-E2(UAA) replicon, which produces core, NS5A and E2 (FIG. 1B). This indicates that HCV E2 is the key protein involved in induction of survival genes, even though core and NS5A may also contribute to activation of NF-κB.

HCV E2 has been reported to trigger transcription of two molecular chaperones GRP94 and GRP78 in the lumen of ER, although the molecular detail of the mechanism involved has not been determined. Our research was focused on GRP94 rather than GRP78, which is also shown to have cytoprotective function because GRP78 has been reported not to activate NF-κB. We found that HCV E2 and GRP94 are partially co-localized in the ER (FIG. 2). Moreover, HCV E2 and GRP94 form a complex, as shown by co-immunoprecipitation analysis. Nevertheless, we do not know whether the GRP94-HCV-E2 complex is involved in the transcriptional activation of GRP78 and GRP94. One plausible hypothesis for the induction of these molecular chaperons is that ‘ER stress’ generated by the production of HCV E2 may trigger the production of GRP78 and GRP94 similarly to the induction of these proteins by the large surface protein of hepatitis B virus (HBV). Investigations into the mechanism of GRP94 induction by HCV E2 are in progress.

Overproduction of GRP94 by HCV may be related to the pathological progress of HCV patients. It is likely that the anti-apoptotic activity of E2 contributes to persistent HCV infections, resulting in chronic hepatitis, by blocking innate and acquired immunity. Moreover, the anti-apoptotic activity may also contribute to generation of HCC. In this respect, it is noteworthy that transgenic mice expressing HCV core-E1-E2 proteins produced larger HCC, compared with control mice or transgenic mice expressing HCV core alone, when they were treated with diethylnitrosamine. Moreover, the HCV transgenic mice liver producing core-E1-E2 underwent significantly less apoptosis than transgenic tissue expressing core only in a Fas-induced apoptosis analysis system. It should be noted that very high levels of GRP94 proteins were observed in the tumor tissues of the HCV patient (FIG. 7C) and that a large portion of GRP94 proteins are redistributed to the edges of cells (FIG. 7C). Interestingly, a similar GRP94 distribution pattern has been reported in other tumor cells. Moreover, the increase in GRP94 level was shown to be correlated with the disease progression of HBV infection; although unrelated to HCV, HBV shows similar pathological progress as HCV. The levels of GRP94 were greatest in HCC patients and lowest in patients with chronic HBV infection, with intermediate levels reported in patients with cirrhosis. This may indicate that the pathological progress of hepatotropic viruses (HBV and HCV) is mediated by GRP94, although it is not clear which HBV protein induces production of GRP94.

Investigations into the function of GRP94 will further our understanding of the pathogenic process after HCV infection and aid the development of anti-HCV drugs that activate defense mechanisms against viral infections.

### Example

The present invention is further explained in more detail with reference to the following examples. These examples, however, should not be interpreted as limiting the scope of the present invention in any manner.

[Materials snd Methods]

Establishment of a Cell Line Expressing Grp94—a Plasmid pCDNA

3.1-GRP94 expressing GRP94 (Hoshino T, Nakaya T, Araki W, Suzuki K, Suzuki T, Mizushima T. Endoplasmic reticulum chaperones inhibit the production of amyloid-beta peptides. Biochem J 2007; 402:581-589) was kindly provided by Dr. Tohru Mizushima (Kumamoto University, Japan). A control plasmid pCDNA 3.1 (10 μg) and the plasmid pCDNA 3.1-GRP94 (10 μg) were transfected into Huh-7 cells by electroporation. From 48 h posttransfection, cells were maintained in DMEM containing G418 (600 μg/ml; Calbiochem). After 3 weeks of selection, G418-resistant cell colonies were pooled and cultivated for further analyses.

Antibodies and chemicals—The monoclonal antibody H52 against E2 was a gift from Dr. Dubuisson at the University of Lille. Actin antibody was purchased from ICN. Antibodies against GRP94, Hsp90, survivin, and IKKα/β were purchased from Santa Cruz. Antibodies against TRAF2 and XIAP were purchased from BD, and 17-AAG was obtained from Sigma Aldrich Co.

Cell culture and transient transfection—Huh-7 cells and 293T cells were grown at 37° C. in Dulbecco's modified Eagle's medium (Gibco) supplemented with antibiotics (penicillin 100 U/ml, streptomycin 10 μg/ml) and 10% fetal bovine serum (Hyclone) in the presence of 6.0% CO₂. FK-E1(UAA) and FK-E2(UAA) replicons, and control (vector), and GRP94 (GRP94-overexpressing) cell lines were grown under the same conditions but with the addition of the antibiotic G418 (600 μg/ml; Calbiochem). The control and spE2 (HCV-E2-expressing cell line) (Lee S H, Kim Y K, Kim C S, Seol S K, Kim J, Cho S, Song Y L, et al. E2 of Hepatitis C Virus Inhibits Apoptosis. J Immunol 2005; 175:8226-8235) were grown under the same conditions but with the addition of the antibiotic hygromycin B (300 (g/ml; Calbiochem). 293T cells were electroporated as previously described (Lee S H, Kim Y K, Kim C S, Seol S K, Kim J, Cho S, Song Y L, et al. E2 of Hepatitis C Virus Inhibits Apoptosis. J Immunol 2005; 175:8226-8235).

Knockdown of GRP94 using siRNA—Duplex siRNAs targeted to GRP94 and TRAF2, and control siRNAs were purchased from Bioneer Inc. (Korea). The siRNA sequences targeting GRP94 (GRP94 siRNA1 and GRP94 siRNA2) were 5′-UGA UGU GGA UGG UAC AGU A dTdT-3′ (SEQ ID NO: 1+dTdT) and 5′-UAC UGU ACC AUC CAC AUC A dTdT-3′ (SEQ ID NO: 2+dTdT). The control siRNA sequence was 5′-CCU ACG CCA CCA AUU UCG UdTdT-3′ (SEQ ID NO: 10). The siRNA sequence targeting TRAF2 (TRAF2 siRNA) was 5′-CAA CCA GAA GGU GAC CUU A dTdT-3 (SEQ ID NO: 11). To transfect siRNA into Huh-7 cells, 100 nM siRNA mixed with 3 (I of Lipofectamine 2000 (Invitrogen) was added to each well of a six-well plate. The cells were harvested 72 hours after the first transfection.

Luciferase assay—Luciferase assays were performed using a luciferase assay kit (Promega), according to the manufacturer's instructions. The luciferase activities in the cells were normalized by the protein concentrations, as determined by the Bradford assay.

Western-blot analysis—Proteins were resolved by 8% or 13.5% SDS-PAGE and transferred to nitrocellulose membranes (Amersham). The membranes were blocked overnight with 5% skimmed milk in Tris-buffered saline (TBS) [20 mM Tris-HCl (pH 7.5), 150 mM NaCl, 0.5% Tween-20] and then incubated with monoclonal antibodies against actin (1:5000), GRP94 (1:1000), Hsp90 (1:1000), TRAF2 (1:500), XIAP (1:1000), survivin (1:500), glyceraldehyde-3-phosphate dehydrogenase (GAPDH) (1:5000) and IKKα/β for 5 hours. A monoclonal anti-human actin antibody was used as a control. Horseradish-peroxidase-conjugated anti-mouse, anti-goat or anti-rabbit immunoglobulin Gs (IgGs) were used as secondary antibodies (1:5000), and bands were visualized by enhanced chemiluminescence (ECL) according to the manufacturer's instructions (Amersham).

Patient Tissue—Human tissue specimens were supplied from the Liver Cancer Specimen Bank supported by National Research Resource Bank Program of the Korea Science and Engineering Foundation in the Ministry of Science and Technology. The consents to utilize the tissue specimens for research purposes were obtained from patients, and the utilization of the specimens for this research was authorized by the Institutional Review Board of College of Medicine, Yonsei University.

Fluorescence Microscopy—For fluorescence microscopic analysis, frozen liver tissues originated from patients with tumors were fixed in 4% paraformaldehyde (PFA) overnight at 4° C. and embedded in OCT (Fisher Scientific) for sectioning. The sections (12 μm) were stained with hematoxylin and eosin (H&E). For immunostaining, sections were fixed in 4% PFA and the antigenic epitopes were exposed by treating with 10 mM citrate buffer and heating in a microwave oven. Sections were incubated in blocking solution (3% bovine serum albumin, 5% horse serum, and 0.5% Tween-20 in phosphate-buffered saline) at room temperature for 4 hours, followed by an additional incubation with monoclonal antibody against E2 (H52) (1:100 dilution) and goat polyclonal antibody against GRP94 (1:100 dilution; Santa Cruz Biotechnology). Specific binding was detected with Alexa 488-labeled anti-mouse and Alexa 594-labeled anti-goat IgGs (Molecular Probes). Fluorescence microscopy was performed as described previously (46). A primary rabbit antibody against GRP94 and a mouse antibody against E2 were used to detect co-localization of the two proteins.

### Example 1

### HCV E2 Augments Expression of Survival Genes Related with NF-κB Activation

The inventors have previously shown that the HCV E2 protein inhibits apoptosis triggered through the mitochondrial pathway (Lee S H, Kim Y K, Kim C S, Seol S K, Kim J, Cho S, Song Y L, et al. E2 of Hepatitis C Virus Inhibits Apoptosis. J Immunol 2005; 175:8226-8235). To determine the molecular basis of the HCV E2 anti-apoptotic activity, we monitored the levels of proteins known to be involved in cell survival and anti-apoptosis processes by Western-blot assays in HCV-E2-expressing cells (spE2, a derivative of the hepatocellular carcinoma cell line Huh-7). To minimize the artificial effects of individual colonies expressing E2, a pool of E2-gene-containing colonies generated by permanent cell line selection methods was used. Of the proteins tested, the levels of the anti-apoptotic proteins XIAP, TRAF2 and FLIP in the spE2 cells were examined by western blot analysis, and the obtaining results are shown in FIG. 1A, indicating that the levels of the anti-apoptotic proteins XIAP, TRAF2 and FLIP increased in the spE2 cells. The levels of phosphorylated IKKα/β, which is a measure of NF-κB activation, were also measured with an antibody against phosphorylated IKKα/β, and the obtained results are shown in FIG. 1A; this is because the anti-apoptotic proteins XIAP, TRAF2 and FLIP are under the control of NF-κB. The level of phosphorylated IKKα/β was also increased in the spE2 cells as shown in FIG. 1A.

Similarly, increased levels of the anti-apoptotic proteins XIAP, TRAF2, and survivin, which are under the control of NF-κB, were observed in Huh-7 cells containing the HCV replicon FK-E2 (UAA) expressing HCV nonstructural proteins (NS3, NS4A, NS4B, NS5A and NS5B), which are required for replication of HCV RNA, and structural proteins (core, E1 and E2), and the obtained immunocytochemical analysis results are shown in FIG. 1B. On the other hand, the levels of these anti-apoptotic proteins in Huh-7 cells containing the HCV replicon FK-E1(UAA) that expresses HCV nonstructural proteins and the structural proteins core and E1, but not E2 were the same as those in control Huh-7 cells without a HCV replicon (FIG. 1B). These data indicate that the levels of the anti-apoptotic proteins XIAP, TRAF2, FLIP and survivin are strongly increased with HCV E2, regardless of the presence of other HCV proteins.

The protein level of IκBα in control Huh-7 cells, Huh-7 cells containing replicons FK-E1 (UAA), and FK-E2(UAA) were measured by Western-blot assays with time; this is because the phosphorylation-dependent degradation of IκBs is an essential step for NF-κB activation after TNF-α treatment. The obtained results were shown in FIG. 1C. As shown in FIG. 1C, similar levels of IκBα proteins were observed in the control Huh-7 cells and in the Huh-7 cells containing FK-E1 (UAA) before TNF-α treatment. Within 5 minutes of TNF-α treatment, IκBα proteins were not detected in the control Huh-7 cells (FIG. 1C). In Huh-7 cells containing the replicon FK-E1(UAA) that expresses NS3-5B, core and E1 proteins, the degradation of IκBα was delayed by up to 10 minutes after treatment with TNF-α (FIG. 1C). These results indicate that one of the HCV proteins encoded by the FK-E1(UAA) replicon blocks degradation of IκBα.

Importantly, the amount of IκBα in untreated cells was lowest in the cells containing the FK-E2(UAA) replicon and expressing HCV NS3-5B, core, E1 and E2 (FIG. 1C). Moreover, the residual IκBα proteins were not detected in the FK-E2(UAA)-containing cells within 5 minutes of TNF-α treatment (FIG. 1C). This indicates that NF-κB may be partially activated in E2-expressing cells, owing to the reduced basal level of IκBα, but that it is fully activated soon after treatment with TNF-α, even in the presence of other HCV proteins. Taken together, these data indicate that HCV E2 may induce expression of anti-apoptotic proteins through activation of NF-κB.

### Example 2

### Overexpression of GRP94 in Cells Expressing HCV E2 Protein

The inventors attempted to determine the mechanism by which HCV E2 activates NF-κB, and focused on a report that suggested that E2 activates the GRP94 promoter. GRP94 is a molecular chaperone in the lumen of the endoplasmic reticulum (ER).

The expression levels and subcellular localization of E2 and GRP94 was investigated by Western-blot analysis (FIG. 3C and FIG. 4C), and immunocytochemical analysis with spE2 (FIG. 2A) and FK-E2(UAA) cells (FIG. 2B). To minimize the artificial effects of individual colonies overexpressing HCV E2, a pool of E2-overexpressing cells (spE2) that had been selected during generation of a permanent cell line were used.

FIGS. 2A and 2B shows the level of GRP94 measured by Immunocytochemical analyses, indicating that higher levels of GRP94 were observed in HCV-E2-expressing cells compared with non-expressing cells. Both E2 and GRP94 were found to be localized in the ER. FIG. 2A shows results of the immunocytochemical analyses of GRP94 and E2 in spE2 cells. Both GRP94 and HCV E2 proteins were localized to the ER. The immunocytochemical analyses were performed with anti-GRP94 and anti-E2 (H52) antibodies. The localizations of GRP94 (TRITC) and E2 (FITC) are shown in red and green, respectively. The yellow regions represent GRP94 and HCV E2 co-localized areas. FIG. 2B shows the results of immunocytochemical analyses of GRP94 and E2 proteins in Huh-7 cells containing the replicon FK-E2(UAA). The localizations of GRP94 (TRITC) and E2 (FITC) are shown in red and green, respectively. Different levels of HCV E2 were observed in cells, which may reflect the different copy numbers of the HCV replicon.

Interestingly, the GRP94 proteins seemed to be co-localized with E2, as indicated by yellow regions in FIGS. 2A and 2B. The intensities of red signals revealed that GRP94 protein levels were increased in the spE2 cells (FIG. 2A) and Huh-7 cells containing the FK-E2(UAA) replicon expressing high levels of E2 (FIG. 2B).

### Example 3

### NF-κB Activation by GRP94

The relationship between increased expression of GRP94 and activation of NF-κB was investigated because both of these biological events are triggered by HCV E2. A cell line that overexpressed GRP94 were established (FIG. 3A). To minimize the artificial effects of individual colonies overexpressing GRP94, a pool of GRP94-overexpressing cells (GRP94) selected out in the process of a permanent cell line generation was used. FIG. 3A shows the levels of NF-κB-related survival gene products in cells overexpressing GRP94. The levels of various proteins in control cells (lane 1) and in GRP94-overexpressing cells (lane 2) were assessed by Western-blot analysis.

Expression levels of the NF-κB-related gene products TRAF2, XIAP and survivin were increased in GRP94-overexpressing cells (FIG. 3A), as seen in HCV-E2-overexpressing cells (FIGS. 1A, 1B). Moreover, the expression levels of IKKα/β, which is also under the control of NF-κB, were markedly increased in GRP94-overexpressing cells. On the other hand, levels of Hsp90, a GRP94-related protein, and actin (a negative control) were not changed in GRP94-overexpressing cells. These results indicate that GRP94 affects NF-κB-related gene expression.

Subcellular localizations of GRP94 and IKKα/β were monitored by immunocytochemical analysis (FIG. 3B). FIG. 3B shows subcellular localizations of GRP94 and IKKα/β in normal and GRP94-overexpressing cells. Immunocytochemical analyses were performed with anti-GRP94 and anti-IKKα/β antibodies. The localizations of GRP94 (FITC) and IKKα/β (TRITC) are shown in green and red, respectively. The yellow regions represent GRP94 and IKKα/β co-localized areas. Panels a-h represent magnified images of cells shown in the upper panel. Results with a typical GRP94-overexpressing cell (white box of upper panel) are shown in panels a-d. A typical cell, which expresses normal levels of GRP94 (pink box of upper panel), is shown in panels e-h. Greater levels of IKKα/β in the filamentous form are observed in cells overexpressing GRP94.

FIG. 3B shows that GRP94 proteins were localized to the ER in cells expressing high and normal levels of GRP94 and IKKα/β. Interestingly, higher levels of IKKα/β were observed in cells expressing higher levels of GRP94 (FIG. 3B), which is consistent with the Western-blot results (FIG. 3A). Interestingly, the GRP94 proteins were partially co-localized with the cytoplasmic IKKα/β proteins in GRP94-overexpressing cells.

The role of GRP94 in NF-κB activation was further investigated by an RNA-interference method using siRNAs against GRP94. siRNAs against GRP94 and a control siRNA were transfected into control and spE2 cells, and the levels of NF-κB-related gene products were monitored by Western-blot analysis (FIG. 3C). FIG. 3C shows effects of siRNAs against GRP94 on the levels of NF-κB-related survival gene products in cells. The levels of various proteins in control cells (lanes 1-3) and in GRP94-overexpressing cells (lanes 4-6) were observed by Western-blot analysis. The normal and GRP94-overexpressing cells were treated with a control siRNA (lanes 1 and 4) or two different siRNAs (lanes 2 and 3 and lanes 5 and 6) before analysis of proteins levels by Western-blot assays.

siRNAs against GRP94 (GRP94 siRNA-1 and GRP94 siRNA-2) partially reduced the levels of GRP94 in normal cells (FIG. 3C) and in GRP94-overexpressing spE2 cells (FIG. 3C) compared with control siRNA. Treatment with siRNA against GRP94 reduced the basal levels of GRP94, XIAP, TRAF2, survivin and IKKα/β in normal cells (FIG. 3A) and those of proteins overshooted by production of HCV E2 (FIG. 3A). By contrast, the level of the negative control actin was not affected by the presence of the GRP94-specific siRNAs (FIG. 3C). There were no reductions in the expression levels of E2 and Hsp90 with siRNA treatment (FIG. 3C). These data indicate that increased expression of NF-κB-related genes is mediated by the overproduction of GRP94, which in turn is induced by HCV E2. In other words, GRP94 is a mediator of anti-apoptotic activity of HCV E2. Curiously, increased expression of GRP78 was consistently observed in the cells treated with siRNAs against GRP94 (FIG. 3C). The inventors speculate that the increase in GRP78, another molecular chaperone in the ER lumen, may compensate for the reduced levels of the molecular chaperone GRP94 by an as-yet-unknown mechanism.

The activation of NF-κB by overproduction of GRP94 was investigated by measuring the activity of a reporter gene (firefly luciferase) under the control of the NE-KB response element (FIG. 4A). FIG. 4A shows the results of NF-κB reporter assay. 293T cells were transfected with pNF-κB-Luc, which encodes the firefly luciferase gene under the control of a NF-κB response element, with control vector plasmid (pCDNA3.1) (lane 3) or with the plasmid pCDNA3.1-GRP94, which produces. GRP94 (lane 4). Control plasmid pRLCMV-Luc (Promega) was also cotransfected to normalize transfection efficiency. After incubation of the cells for 48 hours, cells were further incubated in serum-free medium for 6 hours, mock-treated (lane 1), or treated with 10 ng TNF-α for 12 hours (lane 2), before luciferase activities were measured. Three independent experiments were performed and the relative values (TNF-α-treated vs mock-treated, and GRP94-overexpressed vs mock-expressed) are shown. The bottom panel shows GRP94 protein levels measured by Western-blot analysis with an anti-GRP94 antibody.

As shown in FIG. 4A, overproduction of GRP94 increases NF-κB-dependent luciferase activity by about 3-5-fold compared with the control vector. An increase in NF-κB reporter activity of about 10-fold was observed with TNF-α treatment (FIG. 4A). These data indicate that overproduction of GRP94 activates NF-κB.

The inventors tried to determine the stage of the signaling cascade at which NF-κB is activated by GRP94. First, the role of IKKα/β was investigated because it plays a key role in activation of NF-κB by cytokines such as TNF-α. The effects of BAY11-7082, a specific inhibitor of IKKα/β, on the expression of NF-κB-related gene products were monitored using GRP94-overexpressing and control cell lines, and the obtained results are shown in FIG. 4B. FIG. 4B shows that IKKα/β activity is required for NF-κB activation by GRP94. Effect of abrogation of IKKα/β activity by BAY11-7082 on the expression of NF-κB-related survival genes was monitored by Western-blot analysis; lanes 2 and 4 show results with and lanes 1 and 3 show results without treatment of the drug (200M) for 4 hrs on control (lanes 1 and 2) and GRP94-overexpressing (lanes 3 and 4) cells.

As shown in FIG. 4B, the levels of XIAP, TRAF2, survivin, and IKKα/β were markedly reduced in GRP94-expressing and control cells treated with BAY11-7082. These data indicate that IKKα/β activation is needed for GRP94-induced activation of NF-κB. Curiously, the levels of GRP94 protein were reduced by BAY11-7082 treatment (FIG. 4B). This indicates that expression of GRP94 may also be influenced by NF-κB. If this is the case, GRP94 production and NF-κB activation form a positive feedback loop; however, further experiments are needed to confirm this hypothesis.

We also investigated the role of TRAF2, a TNF-α-receptor-associated protein essential for TNF-α signaling upstream of IKKα/β, in HCV E2 activation of NF-κB. The effects of siRNA against TRAF2 on expression of NF-κB-related gene products were monitored by Western-blot analysis. The obtained results are shown in FIG. 4C. The levels of E2, GRP94, XIAP, TRAF2, survivin, IKKα/β, HSP90, GRP78 and actin were monitored by Western-blot analysis. The control (lanes 1 and 2) and E2-expressing (lanes 3 and 4) cells were treated with a control siRNA (lanes 1 and 3) or a siRNA against TRAF2 (lanes 2 and 4).

As expected, the siRNA against TRAF2 blocked expression of TRAF2 (FIG. 4C). However, there was no reduction in the levels of NF-κB-related proteins (XIAP, survivin, IKKα/β) after treatment with siRNAs against TRAF2 (FIG. 4C). These data indicate that activation of NF-κB by HCV E2 is independent of TRAF2, TNF-α receptor and related proteins because TRAF2 mediates the NF-κB activation through TNFR1, TNFR2 and CD40.

### Example 4

### Activity of GRP94 to Prevent Apoptosis Induced by TRAIL

The role of GRP94 in inhibition of apoptosis during HCV infection was investigated using a siRNA against GRP94 and a control siRNA, the GRP94-overexpressing, and the E2-expressing cell lines (FIG. 5). The strong apoptosis inducer TRAIL was used to mimic the physiological conditions of a virus infection that induces TRAIL expression. Cleavage of PARP, which is a substrate of activated caspase-3/7, was used to monitor the process of apoptosis. The obtained results were shown in FIG. 5, wherein control (lanes 1-4), GRP94-overexpressing (lanes 5-8), and E2-expressing (lanes 9-12) cells were mock-treated (lanes 1, 3, 5, 7, 9 and 11) or treated with 200 ng/ml TRAIL (lanes 2, 4, 6, 8, 10 and 12) for 2 hours after treatment with a control siRNA (lanes 3, 4, 7, 8, 11 and 12) or a siRNA against GRP94 (lanes 1, 2, 5, 6, 9 and 10). The levels of uncleaved PARP, GRP94, HCV E2 and actin were monitored by Western-blot analysis.

After treatment with TRAIL, cleavage of PARP was observed in control cells irrespective of the presence of control siRNA or GRP94 siRNA (FIG. 5). On the other hand, cleavage of PARP was blocked in the GRP94-overexpressing (FIG. 5) and in the HCV-E2-expressing cells (FIG. 5).

This indicates that GRP94 overexpression can inhibit TRAIL-induced apoptosis. Importantly, these anti-apoptotic activities of GRP94 and HCV E2 were abrogated by treatment with siRNA against GRP94 (FIG. 5). This effect is consistent with inhibition of NF-κB-related gene expression by siRNAs against GRP94 (FIG. 3C). These data indicate that the anti-apoptotic activity of HCV E2 is mediated by overexpression of GRP94. It should be noted that basal expression of GRP94 was not sufficient to block apoptosis by TRAIL (FIG. 5). This may indicate that basal levels of anti-apoptotic proteins such as XIAP, FLIP and survivin in normal cells are too low to block apoptotic signals generated by TRAIL even with basal levels of GRP94 (FIGS. 1A and 1B).

### Example 5

### GRP94 is Needed for Blockage of Apoptosis Induced by TRAIL in HCV-Infected Cells

To confirm that the anti-apoptotic activity of E2 is induced by GRP94 and NF-κB-related gene products in HCV-infected cells, the levels of GRP94, XIAP, TRAF2, FLIP and Bcl-xL were monitored using a HCV-infection system. The infectious JFH1 strain of HCV (Wakita T, Pietschmann T, Kato T, Date T, Miyamoto M, Zhao Z, Murthy K, et al. Production of infectious hepatitis C virus in tissue culture from a cloned viral genome. Nat Med 2005; 11:791-796) and a JFH1 derivative known as JFH5a-Rluc with a reporter gene (Renilla luciferase) in the NS5A of HCV (FIG. 6A) that is suitable for quantification of HCV infection (Kim C S, Jung J H, Wakita T, Yoon S K, Jang S K. Monitoring the Antiviral Effect of Alpha Interferon on Individual Cells. J. Virol. 2007; 81:8814-8820) were used. FIG. 6A schematically represents JFH15a-Rluc containing the Renilla luciferase gene in NS5A of the infectious HCV strain JFH1.

Three days after infection, Renilla luciferase activity was greatly increased in the Huh-7 cells infected with JFH 5a-Rluc, indicating that the infection and replication of HCV occurred properly. FIG. 6B shows the levels of GRP94 and NF-κB-related survival gene products in control and HCV (JFH 5a-Rluc)-infected Huh-7 cells, as monitored by Western-blot analysis. The levels of GRP94, XIAP, TRAF2, FLIP, Bcl-xL, phospho-IκBα, IκBα and actin were monitored by Western-blot analysis. Western-blot analysis of GRP94 and NF-κB-related proteins showed that the levels of cell-survival-related gene products (XIAP, FLIP and Bcl-xL) were greatly increased in the HCV-infected cells (FIG. 6B). Considering that HCV E2 induces GRP94 production and that overexpressed GRP94 activates NF-κB, the induction of these proteins is attributed to the overproduction of GRP94 and NF-κB activation, as indicated by the increased levels of GRP94 and phosphorylated IκBα, and the reduced level of IκBα (FIG. 6B).

Consistent with this hypothesis, immunocytochemical analysis showed greatly increased levels of GRP94 in the Huh-7 cells infected with JFH 5a-Rluc (FIG. 6C). FIG. 6C shows results of immunocytochemical analyses of GRP94 and HCV E2 in HCV-infected cells. A confocal immunofluorescence microscope was used to detect the proteins GRP94 (red) and E2 (green) in cells uninfected and infected with HCV (JFH 5a-Rluc). Note that higher levels of GRP94 are observed in the cells infected with HCV compared with the uninfected cells (FIG. 6C).

The apoptosis-blocking effects of GRP94 in HCV-infected cells were observed with TRAIL, which induces apoptosis of cancer cells and virus-infected cells with or without treatment of a siRNA against GRP94, and the obtained results are shown in FIG. 6D. FIG. 6D shows effects of TRAIL and a siRNA against GRP94 on infectivity of HCV. Huh-7 cells were transfected with a control siRNA (lanes 1, 2, 5 and 6) and a siRNA against GRP94 (lanes 3, 4, 7 and 8) and incubated for 48 hours. The siRNA-treated cells were mock-infected (lanes 1-4) or infected with HCV (JFH 5a-Rluc) (lanes 5-8) and incubated for 24 hours. After infection, the cells were mock-treated (lanes 1, 3, 5 and 7) or treated with TRAIL (lanes 2, 4, 6 and 8) for 18 hours. These cells were used to monitor protein levels (upper panel) and virus infection (lower panel). The levels of PARP, GRP94 and actin were monitored by Western-blot analysis. The HCV infectivity levels were assessed by measuring luciferase activities. Experiments were performed six times with triplicate samples each, and mean and standard deviation values of the experiments are shown as columns and lines, respectively.

As shown in FIG. 6D, the cleavage of PARP in mock-infected cells was observed in TRAIL-treated cells, even in the presence of basal levels of GRP94. Marked changes in PARP cleavage efficiency were observed in HCV-infected cells. There were almost no reductions in the levels of PARP cleavage in HCV-infected cells, even after TRAIL treatment (FIG. 6D). This indicates that one of the HCV proteins blocks TRAIL-induced apoptosis in HCV-infected cells. The blockade of apoptosis in HCV-infected cells was nullified by knocking down of GRP94, as shown by monitoring the PARP cleavage levels in GRP94-specific siRNA-treated HCV-infected cells (FIG. 6D).

The role of GRP94 in HCV infection was monitored by measuring the infectivity of the HCV strain JFH 5a-Rluc in the presence of TRAIL. Infectivity of HCV was reduced by about 20% with TRAIL treatment (FIG. 6D). Infectivity of HCV was reduced by about 10% with GRP94-specific siRNA treatment (FIG. 6D). The most marked reduction in HCV infectivity (almost 70%) was observed in cells treated with TRAIL and the siRNA against GRP94 (FIG. 6D). These data indicate that GRP94 may play a key role in HCV infection and establishment of chronic hepatitis.

### Example 6

### Increased Level of GRP94 Protein is Observed in the HCV-Infected Liver Cells of HCV Patients

The pathological relevance of GRP94 overproduction in HCV-infected hepatocyte was investigated using liver tissue samples from HCV patients that were obtained from the Liver Cancer Bank in Korea (http://www.liverca.com/).

Clinical background data are summarized in Tables 1 and 2.

TABLE 1
Description of Normal Liver

| | | | Pathologic Status | Viral Marker |
|---|---|---|---|---|
| Case No. | Age | Gender | of Adjacent Liver | (HCV) |
| 339 | 37 | M | Within normal limit | Anti-HCV negative |
| 398 | 53 | M | Within normal limit | Anti-HCV negative |

TABLE 2
Clinical background of the HCC patients with HCV infection

| | | | | | | | Pathologic Status | Preventent |
|---|---|---|---|---|---|---|---|---|
| Case No. | Age | Sex | Diagnosis | Tumor Size | Grade | Necrosis (%) | of Adjacent Liver | Cause |
| 138 | 67 | M | Hepatocellular carcinoma | 3.0 | 2 | 0 | Chronic hepatitis | HCV |
| 388 | 61 | M | Hepatocellular carcinoma | 6.4 × 6.5 | 2 | 0 | Chronic hepatitis | HCV |
| 311 | 66 | F | Hepatocellular carcinoma | 6.5 × 5.5 | 2 | 0 | Chronic hepatitis | HCV |
| 292 | 69 | M | Hepatocellular carcinoma | 5.5 × 4.5 | 2 | 70 | Chronic hepatitis | HCV |

Overall morphologies of normal liver, HCV-infected liver, and HCV-positive cancerous liver tissues were observed by H&E staining (FIG. 7A). FIG. 7A visualizes liver cells from a normal liver (left panel) and HCV patients (middle and right panels) by H&E staining. The donor of the liver tissue shown in the middle panel is a chronic HCV patient. The donor of the liver tissue shown in the right panel is HCC patient infected with HCV. The cancerous part of the liver was analyzed. Abnormal morphologies of liver tissues were observed in HCV-infected and cancerous liver tissues (FIG. 7A).

HCV was confirmed in liver tissues from a chronic HCV patient and from a HCC patient chronically infected with HCV by Western-blot analysis with an antibody against HCV E2, and the obtained results are shown in FIG. 7B. As shown in FIG. 7B, increased levels of GRP94 were detected in these patient tissues. Expression levels of the anti-apoptotic protein XIAP, which is under the control of NE-KB, were greatly increased in the liver tissues from HCV patients and from HCC patients infected with HCV compared with levels in normal liver tissue (FIG. 7B).

The relationship between HCV E2 and production of GRP94 was further analyzed by immunohistochemical analysis of liver tissues (FIG. 7C). FIG. 7C shows the results of immunohistochemical analyses of liver tissues from normal (panels a, b, g, h, m, n, s, and t), HCV-infected (c, d, I, j, o, p, u, and v), HCC (e, f, k, l, q, r, w, and x) donors. HCV E2 (panels a-f) and GRP94 (panels g-l) proteins are shown in green and red, respectively. The nuclei of cells were visualized by DAPI staining shown in blue (panels m-r). The merged images are shown in panels s to x. Panels b, d, f, h, j, l, n, p, r, t, v, and x are magnified images of white boxes in panels a, c, e, g, i, k, m, o, q, s, u, and w, respectively.

As expected, no HCV E2 protein was observed in the normal liver tissue (FIG. 7C), and a low level of GRP94 was detected in the tissue (FIG. 7C, panels a, b, g, h, s, and t). Some cells in the liver tissues of the HCV patient (FIG. 7C, panels c, d, i, j, u, and v) and the HCV-positive HCC patient (FIG. 7C, panels e, f, k, l, w, and x) revealed HCV E2 protein. Increased levels of GRP94 proteins were also observed in some cells of the liver tissues of the HCV patient (FIG. 7C) and the HCV-positive HCC patient (FIG. 7C). Importantly, the increased levels of GRP94 coincided with presence of HCV E2 in the patients' liver cells (FIG. 7C). These results indicate that GRP94 expression is increased in HCV-infected cells of the patients and that the GRP94 activity seen in cell-culture systems (induction of survival gene products through activation of NF-κB) is likely to occur in HCV patients.

1-15. (canceled)

16. A method of identifying an antiviral agent or a drug for inhibiting virus infection or preventing or treating a disease caused by virus infection, comprising the following steps of:
contacting a candidate compound to a biological sample from mammal;
measuring the level of expression of GRP 94, or activity of GRP 94;
and comparing the level measured in the biological sample contacted with the candidate compound to that measured in the biological sample without contacting with the candidate compound, wherein the candidate compound is determined as the drug for inhibiting virus infection or preventing or treating a disease caused by virus infection, when the level measured in the biological sample contacted with the candidate compound is decreased compared with that measured in the biological sample without contacting with the candidate compound.

17. The method according to claim 16, wherein the virus is one or more selected from the group consisting of hepatitis viruses.

18. The method according to claim 17, wherein the virus is Hepatitis C Virus (HCV) or Hepatitis B Virus (HBV).

19. The method according to claim 16, wherein the disease is one or more selected from the group consisting of inflammation, and cancer.

20. The method according to claim 16, wherein the disease is one or more selected from the group consisting of hepatitis, cirrhosis, and liver cancer.

21. The method according to claim 16, wherein the biological sample is a cell, a tissue, an extract of cells or tissues, or a body fluid.

22. The method according to claim 21, wherein the biological sample is a hepatocyte, or a virus-infected cell or tissue.
